(19) United States
(12) Patent Application Publication
(10) Pub. No.: US 2026/0144652 A1
(43) Pub. Date: May 28, 2026

(54) SWING DYNAMICS OF A PROSTHETIC OR ORTHOTIC DEVICE

(71) Applicant: Össur Iceland ehf, Reykjavik (IS)

(72) Inventors: Selina Zwerver, Reykjavik (IS); David Jonsson, Reykjavik (IS); Arni Einarsson, Reykjavik (IS)

(21) Appl. No.: 19/397,496

(22) Filed: Nov. 21, 2025

Related U.S. Application Data
(60) Provisional application No. 63/725,288, filed on Nov. 26, 2024.

(51) Int. Cl. A61F 2/64; A61F 2/70; A61F 5/01

(52) U.S. Cl. CPC A61F 2/64; A61F 5/0123; A61F 2002/701; A61F 2002/704; A61F 2005/0155

(57) ABSTRACT

A prosthetic or orthotic device is described for implementation of a locomotion activity. The prosthetic or orthotic device may cause an actuator to perform a provide a first torque. Based on the first torque and one or more values of one or more parameters, the prosthetic or orthotic device may perform a first portion of a flexion action. Subsequent to or during performance of the first portion of the flexion action, the prosthetic or orthotic device may determine an angle associated with the prosthetic or orthotic device. Based on the angle, the prosthetic or orthotic device may perform an adjustment of the one or more parameters. Based on the adjustment, the prosthetic or orthotic device may perform a second portion of the flexion action. For example, the prosthetic or orthotic device may perform the second portion while gradually increasing the one or more values.

### CROSS-REFERENCE TO RELATED APPLICATIONS

The present application claims priority benefit to U.S. Provisional Application No. 63/725,288, filed Nov. 26, 2024, which is hereby incorporated by reference in its entirety. Any and all applications for which a foreign or domestic priority claim is identified in the Application Data Sheet as filed with the present application are incorporated by reference under 37 CFR 1.57 and made a part of this specification.

### BACKGROUND

### Field

The present application relates to features associated with a control system for managing operation of a prosthetic or orthotic device (e.g., a lower-limb prosthetic device, a motorized prosthetic or orthotic device, etc.).

### Description of the Related Art

Prosthetic or orthotic devices (e.g., a powered knee prosthetic) may include powered actuators that may generate net positive mechanical power. The generation of power by the prosthetic or orthotic devices can enable the prosthetic or orthotic devices to more closely match human limb joint characteristics.

There are several disadvantages to traditional prosthetic or orthotic devices. Such traditional prosthetic or orthotic devices may have difficulty in synchronizing the performance and operation of the traditional prosthetic or orthotic devices to the intent of the users due to the manner in which the control of the motor of the traditional prosthetic or orthotic devices occurs. Further, the velocity of traditional prosthetic or orthotic devices may be limited such that the traditional prosthetic or orthotic devices may not be able to match a desired velocity (e.g., a walking speed of a user).

### SUMMARY

The present disclosure describes systems, methods, and apparatuses related to a prosthetic or orthotic device that may include a first segment, a second segment coupled to the first segment at a joint, an actuator coupled to the first segment and the second segment and configured to control motion of the second segment relative to the first segment, and a controller. The controller may be configured to and/or may cause an actuator to produce a first torque based on one or more parameters of the controller. A first portion of a flexion action may be performed by the prosthetic or orthotic device based on the first torque. The first torque may progressively reduce during performance of the flexion action. The controller may be further configured to and/or may determine a first angle associated with the joint satisfies a first threshold. The controller may be further configured to and/or may perform a first adjustment of the one or more parameters based on determining the first angle satisfies the first threshold and performance of a toe-off assist action. The controller may be further configured to and/or may cause the actuator to produce a second torque based on the first adjustment. A second portion of the flexion action may be performed by the prosthetic or orthotic device based on the second torque.

In some cases, the controller may be further configured to and/or may determine a velocity associated with the prosthetic or orthotic device satisfies a second threshold. Further, the controller may be further configured to and/or may perform a second adjustment of the one or more parameters based on determining the velocity satisfies the second threshold. The controller may be further configured to and/or may cause the actuator to produce a third torque based on the second adjustment. At least a portion of an extension action may be performed by the prosthetic or orthotic device based on the third torque.

In some cases, the velocity may include and/or may be a rotational velocity of the first segment.

In some cases, the controller may be further configured to and/or may determine a velocity associated with the prosthetic or orthotic device satisfies a second threshold. Further, the controller may be further configured to and/or may determine a first portion of an extension action is performed by the prosthetic or orthotic device. Further, the controller may be further configured to and/or may perform a second adjustment of the one or more parameters based on determining the velocity satisfies the second threshold and based on determining the performance of the first portion of the extension action. The controller may be further configured to and/or may cause the actuator to produce a third torque based on the second adjustment. A second portion of the extension action may be performed by the prosthetic or orthotic device based on the second adjustment.

In some cases, the controller may be further configured to and/or may determine a velocity associated with the prosthetic or orthotic device. Further, the controller may be further configured to and/or may perform a second adjustment of the one or more parameters based on the velocity. The controller may be further configured to and/or may cause the actuator to produce a third torque based on the second adjustment. At least a portion of an extension action may be performed by the prosthetic or orthotic device based on the second adjustment.

In some cases, the controller may be further configured to and/or may determine initiation of a swing phase of a locomotion activity. The swing phase may include performance of the flexion action and performance of an extension action. Causing the actuator to produce the first torque may be based on determining the initiation of the swing phase.

In some cases, the flexion action may be associated with a first time period. The extension action may be associated with a second time period. The second time period may exceed the first time period.

In some cases, the one or more parameters may include and/or may be one or more gains.

In some cases, the first adjustment may include and/or may be a progressive adjustment of the one or more parameters over time. The prosthetic or orthotic device may perform at least a portion of a flexion action during performance of the first adjustment.

In some cases, the flexion action may be associated with different rates of adjustment of the one or more parameters.

In some cases, the first adjustment may include and/or may be a gradual increase to one or more values of the one or more parameters.

In some cases, the controller may be further configured to and/or may determine initiation of a swing phase of a first locomotion activity associated with a first velocity. The swing phase of the first locomotion activity may include performance of the flexion action and performance of an extension action. The first adjustment may be based on determining the initiation of the swing phase of the first locomotion activity. Further, the controller may be further configured to and/or may determine initiation of a swing phase of a second locomotion activity associated with a second velocity. Further, the controller may be further configured to and/or may perform the first adjustment based on determining the initiation of the swing phase of the second locomotion activity.

In some cases, the prosthetic or orthotic device may be associated with a stance phase and a swing phase. The stance phase may include a heel strike portion, a foot flat portion, a mid-stance portion, and a toe-off assist portion. The swing phase may include an initial swing portion, a mid-swing portion, and a terminal swing portion. Further, the controller may be further configured to and/or may determine a second angle associated with the joint satisfies a second threshold. Further, the controller may be further configured to and/or may perform a second adjustment of the one or more parameters based on determining the second angle satisfies the second threshold. The controller may be further configured to and/or may cause the prosthetic or orthotic device to perform the terminal swing portion based on the second adjustment.

In some cases, the second adjustment may cause the actuator to maintain a fixed position.

In some cases, the prosthetic or orthotic device may include a control system. The control system may include one or more sensors and the controller. The controller may be further configured to and/or may obtain sensor data from the one or more sensors. The controller may be further configured to and/or may determine the first angle based on the sensor data.

In some cases, the controller may be further configured to and/or may control the actuator based on the first adjustment.

In some case, the first segment may include and/or may be a proximal connector. The proximal connector maya be configured to and/or may attach the prosthetic or orthotic device to a thigh of a trans-femoral amputee.

In some cases, the controller may be further configured to and/or may determine a velocity associated with the thigh satisfies a second threshold. Further, the controller may be further configured to and/or may perform a second adjustment of the one or more parameters based on determining the velocity satisfies the second threshold. The controller may be further configured to and/or may cause the actuator to produce a third torque based on the second adjustment. At least a portion of an extension action may be performed by the prosthetic or orthotic device based on the second adjustment.

The present disclosure further describes systems, methods, and apparatuses related to a non-transitory computer readable medium having instructions stored thereon. Execution of the instructions by a controller may cause the controller to cause an actuator to produce a torque based on one or more parameters of a controller. A prosthetic or orthotic device may include a motorized knee, the actuator, and the controller. The controller may cause the motorized knee to perform a first portion of a flexion action based on the torque. The torque may progressively reduce during performance of the flexion action. Execution of the instructions by the controller may further cause the controller to determine a first angle associated with a knee joint of the motorized knee satisfies a first threshold. Execution of the instructions by the controller may further cause the controller to perform a first adjustment of the one or more parameters based on determining the first angle satisfies the first threshold and performance of a toe-off assist action. The controller may cause the motorized knee to perform a second portion of the flexion action based on the first adjustment.

The present disclosure further describes systems, methods, and apparatuses related to a control system for controlling a prosthetic or orthotic device. The control system may include a controller, a shank segment, and a motorized knee at a knee joint that may be configured to attach to attach to a thigh of a trans-femoral amputee. The controller may be further configured to and/or may cause an actuator to produce a torque based on one or more parameters of the controller. The controller may be further configured to and/or may cause the motorized knee to perform a first portion of a flexion action based on the torque. The torque may progressively reduce during performance of the flexion action. Further, the controller may be further configured to and/or may determine a first angle associated with a knee joint of the motorized knee satisfies a first threshold. Further, the controller may be further configured to and/or may perform a first adjustment of the one or more parameters based on determining the first angle satisfies the first threshold and performance of a toe-off assist action. The controller may be further configured to and/or may cause the motorized knee to perform a second portion of the flexion action based on the first adjustment.

### BRIEF DESCRIPTION OF THE DRAWINGS

FIG. 1A illustrates one embodiment of a prosthetic or orthotic device.

FIG. 1B illustrates another embodiment of a prosthetic or orthotic device.

FIG. 2 is a block diagram of the main hardware components of a prosthetic or orthotic device.

FIG. 3 is a schematic representation of a control system architecture for a prosthetic or orthotic device.

FIG. 4 is a block diagram of an impedance controller used to control an actuator of a prosthetic or orthotic device.

FIG. 5 is a diagram of a gait cycle.

FIG. 6 is a diagram indicating swing dynamics of a prosthetic or orthotic device associated with a gait cycle.

FIG. 7 is a flow diagram depicting example interactions for operating a prosthetic or orthotic device.

### DETAILED DESCRIPTION

In traditional systems, prosthetic or orthotic devices (e.g., powered knee prosthetics) may operate according to a gait cycle (e.g., a gait cycle of a walking locomotion activity). For example, the gait cycle may include a stance phase and a swing phase for the prosthetic or orthotic devices.

A prosthetic or orthotic device may provide and/or cause active movement (e.g., may cause performance of a flexion action or an extension action) and/or passive resistance according to and during the gait cycle. For example, a control system of the prosthetic or orthotic device may provide a control signal to an actuator of the prosthetic or orthotic device based on one or more parameters (e.g., one or more gains) of the control system. In response to the control signal, the actuator may actuate and may cause the prosthetic or orthotic device to perform a flexion action or an extension action.

In traditional systems, the prosthetic or orthotic devices may perform various operations during portions of the gait cycle. During a first portion (e.g., a toe-off portion) of the gait cycle, the control system of the prosthetic or orthotic device may provide a control signal to an actuator of the prosthetic or orthotic device to propel a user forward. For example, during the first portion of the gait cycle, the control system may provide a control signal causing a foot portion of the prosthetic or orthotic device to lift from a ground surface. During a second portion (e.g., an initial swing portion) of the gait cycle, the control system may increase a torque provided by the actuator such that the prosthetic or orthotic device simulates a free moving hinge. During a third portion (e.g., a mid-swing portion) of the gait cycle, the control system may determine an angle of the prosthetic or orthotic device (e.g., an angle of a joint of the prosthetic or orthotic device) and a velocity of the prosthetic or orthotic device and may brake movement of the prosthetic or orthotic device based on the angle and the velocity. For example, during the third portion of the gait cycle, the control system may cause braking of the flexion action and may cause initiation of an extension by the prosthetic or orthotic device. During a fourth portion of the gait cycle (e.g., which may correspond to a transition between the third portion and a fifth portion), the control system may increase a torque provided by the actuator such that the prosthetic or orthotic device simulates a free moving hinge. During a fifth portion (e.g., a terminal swing portion) of the gait cycle, the control system may determine an angle of the prosthetic or orthotic device (e.g., an angle of a joint of the prosthetic or orthotic device) may brake movement of the prosthetic or orthotic device based on the angle and the velocity. For example, the control system may control a position of the actuator such that the actuator maintains a fixed position at a particular angle (e.g., as the prosthetic or orthotic device approaches full extension).

In traditional systems, the operation of the prosthetic or orthotic device may be adjusted according to the determined velocity by adjusting an angle of a joint associated with the prosthetic or orthotic device. For example, a target flexion angle of a joint associated with the prosthetic or orthotic device may be based on a walking speed of a user.

Use of a prosthetic or orthotic device operating in such a manner may be limited to particular operations (e.g., given the limits of the target flexion angle or the maximum target flexion angle such that the velocity and the target flexion angle may be mismatched). While such a prosthetic or orthotic device may provide adequate performance in some specific situations, use of such a prosthetic or orthotic device may not be capable of generalization. For example, the operation strategy may not be synchronized with a desired operation (e.g., a desired walking speed). This may lead to an undesirable user experience as it may cause the operation of the prosthetic or orthotic devices to feel robotic, slow, uncomfortable, etc. For example, in traditional systems, the prosthetic or orthotic devices may not feel and/or may not be responsive to the user.

Furthermore, in traditional systems, by adjusting the operation of the prosthetic or orthotic devices according to the determined velocity, an angle of a joint associated with the prosthetic or orthotic device may increase as a velocity of the prosthetic or orthotic devices increases. This may lead to an undesirable user experience as it may cause the movement of the prosthetic or orthotic device to increase as a duration of the swing phase during the gait cycle decreases (e.g., due to the increased velocity) which may cause the operation of the prosthetic or orthotic device to feel robotic, slow, uncomfortable, etc.

To address these or other concerns, disclosed herein is a control system for operating a prosthetic or orthotic device according to one or more parameters (e.g., one or more dynamic parameters). For example, the one or more parameters may include one or more gains of a controller of the control system. The control system may adjust the one or more parameters to provide an improved performance of the prosthetic or orthotic device. For example, the control system may adjust the one or more parameters to provide an improved user experience (e.g., a smoother user experience). In some cases, the control system may adjust a manner in which the one or more parameters are adjusted (e.g., the degree to which the one or more parameters are adjusted) and/or a timing of the adjustments of the one or more parameters. For example, the control system may perform multiple adjustments of the one or more parameters during performance of a flexion action by the prosthetic or orthotic device.

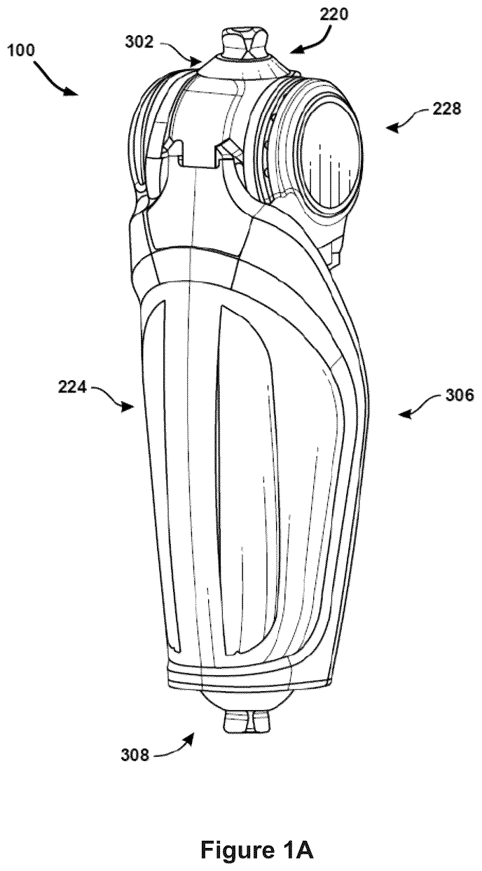

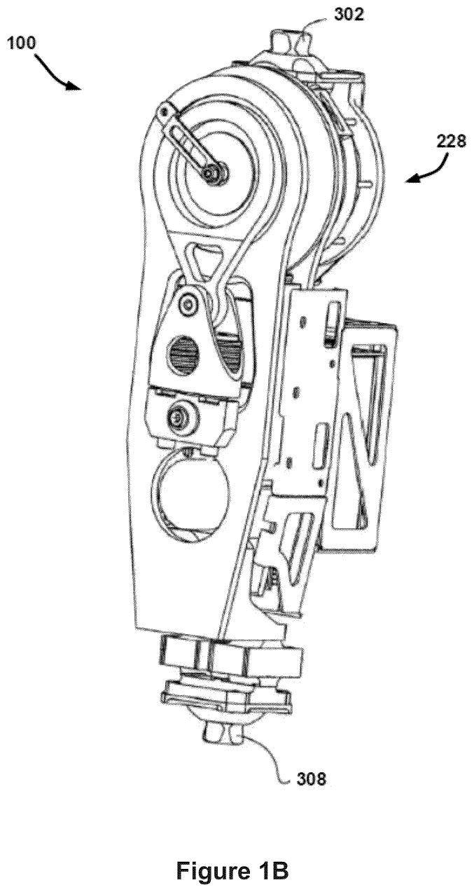

FIGS. 1A and 1B each illustrate an example prosthetic or orthotic device 100 (e.g., a powered knee prosthetic, a processor controlled powered knee prosthetic, a single actuated prosthetic or orthotic device, an active prosthetic or orthotic device, etc.). The prosthetic or orthotic device 100 illustrated in FIGS. 1A and 1B implement a knee joint and may be connected to a user's residual limb, such as a thigh or a stump of an above-knee amputee, through a socket (not shown) via the proximal connector 302 on its proximal segment 220. The proximal connector 302 may be pyramidal, rectangular, or cylindrical in shape. In the examples of FIGS. 1A and 1B, the prosthetic or orthotic device 100 is a lower-limb prosthetic device comprising structures to replace the anatomical structure of the human knee and upper part of the shank. The shank may include the anatomical structure below the knee and above the ankle.

The proximal connector 302 may be coupled to an actuator 228, which can rotate with respect to a body 306. In some cases, the actuator 228 may be motorized. Rotation of the actuator 228 may cause rotation of the proximal connector 302 with respect to the body 306, and vice versa. In some aspects, the body 306 may be on or form a shank portion of the prosthetic or orthotic device 100. The body 306 may include or house electronic components, sensors, etc. (not shown) or other components used to operate the prosthetic or orthotic device 100, although in some aspects these components may be located elsewhere, such as on a peripheral device or within remote components. In some cases, the prosthetic or orthotic device 100 may be connected to a prosthetic or orthotic ankle or foot (not shown) via the distal connector 308 located on a distal segment 224 of the prosthetic or orthotic device 100. The prosthetic or orthotic device 100 may be connected with or include a prosthetic hip, prosthetic thigh, prosthetic foot, prosthetic ankle, or the like.

Example prosthetic or orthotic devices are described in U.S. Pub. No. 2009/0299480, filed Jul. 7, 2009, entitled “Joint Actuation Mechanism for a Prosthetic or Orthotic Device Having a Compliant Transmission,” U.S. Pub. No. 2011/0125290, filed Feb. 2, 2011, entitled “Reactive Layer Control System For Prosthetic And Orthotic Devices,” each of which describes various embodiments and features related to prosthetic or orthotic devices and each of which is hereby incorporated herein by reference in its entirety.

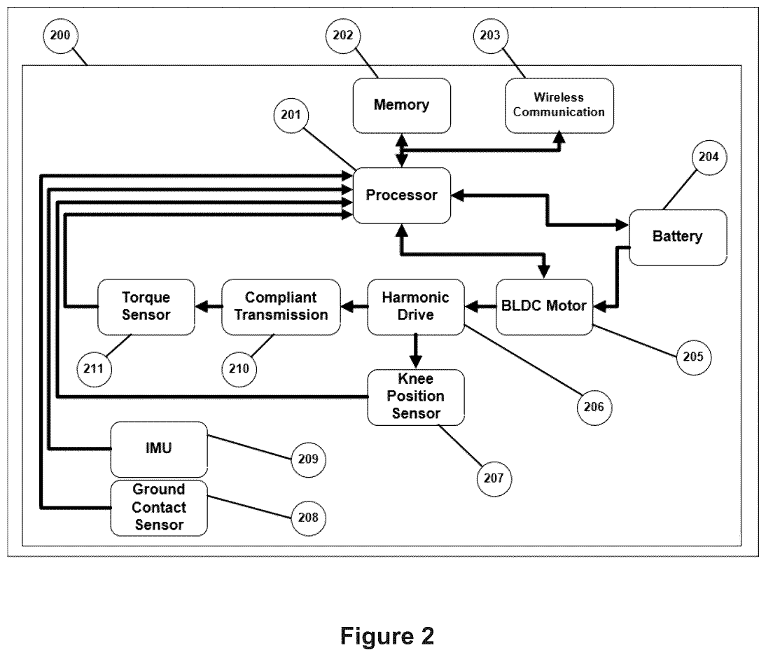

FIG. 2 illustrates a block diagram 200 of example hardware components (e.g., the building blocks) for a prosthetic or orthotic device 100, such as the examples provided in FIGS. 1A and 1B. The prosthetic or orthotic device 100 may use a brushless direct current motor 205 combined with a harmonic drive 206 and a compliant transmission element 210. Alternatively, or in addition, the prosthetic or orthotic device 100 may use one or more alternating current motors and/or brushed motors. The processor 201 may connect most of the other building blocks together. The processor 201 may operate in such a way as to create a cohesive ensemble from the individual components and modules. The processor 201 is in operational communication with a memory 202, where information for the correct system operation can be stored and retrieved. Additionally, or alternatively, the processor 201 manages the wireless communication module 203 to exchange information with external devices, such as mobile phones or tablets, where various types of applications can be used to retrieve information from the prosthetic or orthotic device 100, trigger specific functions, or provide new data to be stored in the memory 202.

The prosthetic or orthotic device 100 may include a battery 204 to power the electronic components, such as the various sensors 207, 208, 209, and 211, the processor 201, and the brushless direct current motor 205. Various types of battery technologies can be used to power the control system of the prosthetic or orthotic device 100. However, in some embodiments, the mobile nature of the system may limit the weight of a battery or power system. Thus, battery technologies providing high power and energy densities may be better suited for this type of application. Additionally, the prosthetic or orthotic device 100 may use secondary type battery technologies to reduce the operating costs of these devices by allowing the battery to be recharged, as well as reduce the environmental impact related to the device's operation.

Embedded sensors are integrated in the basic components of the system hardware. The embedded sensors may be used as input sources for implementing the various control loops and intent management functions. In some embodiments, the prosthetic or orthotic device 100 may use a ground contact sensor to monitor the interaction between the user wearing the prosthetic or orthotic device 100 and the immediate environment. Various constructions can be adopted to implement the ground contact sensor, such as, but not limited to, loadcells, optical displacement sensors, strain gauges, magnetic displacement sensors, inductive displacement sensors, capacitive displacement sensors, pressure sensors, and piezoelectric load sensors. In one non-limitative embodiment of a ground contact sensor system, the ground contact sensor array disclosed in U.S. Provisional Application No. 62/894,442 filed Aug. 30, 2019 and U.S. Nonprovisional application Ser. No. 17/638,493, which was filed Feb. 25, 2022, can be used to provide sensing capabilities to segregate between heel load and toe load due to sensor mechanical properties and use of a sensor array, which is hereby incorporated by reference in its entirety.

In some embodiments, a load sensor assembly may be located at a distal end of the prosthetic or orthotic device 100 near the distal connector 308. The load sensor assembly may have one or more load sensors (e.g., one load sensor, five load sensors, seven load sensors, more than seven load sensors, or any number in between)). The locations of the sensors may represent different load-bearing areas of the prosthetic device 100. For example, the load-bearing surface may be a bottom of a prosthetic or orthotic foot or an ankle. For example, the prosthetic or orthotic device 100 may have two posterior load sensors located towards the rear of the assembly that represent the posterior portion/area of a prosthetic or orthotic foot and have two anterior load sensors located towards the front of the assembly that represent the anterior portion/area of the prosthetic or orthotic foot. More specifically, the two posterior load sensors may represent the heel of the prosthetic or orthotic foot, while the two anterior load sensors may represent the toes of the prosthetic or orthotic foot. In some embodiments, the prosthetic or orthotic device 100 may have three mid-section sensors that represent the mid-section of the prosthetic or orthotic foot. Likewise, the sensor assembly may have one or more sensors that represent the medial (or lateral) side of a prosthetic or orthotic foot. It should be understood that any number of sensors (e.g., one sensor, two sensors, three sensors, more than three sensors, or any number in between) may be used to represent different load-bearing areas, and that some of the load sensors may represent multiple load-bearing areas (e.g., the posterior-lateral area/portion of a prosthetic or orthotic foot).

The prosthetic or orthotic device 100 may integrate one or more Inertial Measurement Units (IMU) to measure the kinematics of the user's residual limb or the prosthetic device. In some embodiments, the prosthetic or orthotic device 100 may include a knee position sensor or another type of sensor that can be used to measure knee joint kinematics directly, which are the result of the interaction between the user, the environment, and the actuator. In some embodiments, the prosthetic or orthotic device 100 may have a knee angle sensor (e.g., an encoder).

In some embodiments, the actuator may include the brushless direct current motor 205, the harmonic drive 206 transmission and the compliant transmission element 210. The actuator may alternatively be separate from and work together with the brushless direct current motor 205, the harmonic drive 206 transmission and the compliant transmission element 210 to control the prosthetic or orthotic device 100.

In some embodiments, the prosthetic or orthotic device 100 may include a torque sensor that may directly measure the interactions occurring between the prosthetic or orthotic device 100 and the user, providing a direct measure of the system kinetics. The sensor described above may be functionally connected with the processor 201. The processor 201 may retrieve the information from the connected sensors and further process it based on the definition of the detection and control processes.

In some embodiments, the brushless direct current motor 205 is functionally connected to and controlled by the processor 201 based on the outcome of the data processing performed in firmware. As mentioned above, the motor-transmission combination, or any other type of actuator, may implement the application-level strategies through motion control of the actuator. Implementation of the motion control strategies, combined with environmental and user interactions may cause a general system response that can be measured by the sensors, hence closing the feedback control loop.

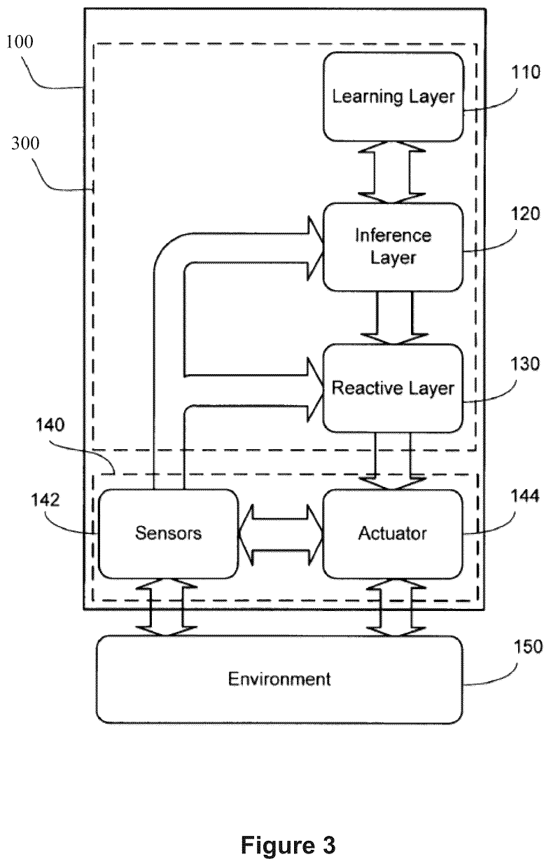

The processor 201 can be leveraged to implement a layered control system. Use of a layered control system architecture for the implementation of the application-level control mechanisms advantageously allows for simple and efficient organization of the data flow in the control system, as well as the creation of a data abstraction model that efficiently divides the tasks between the modules that are cohabiting in the device firmware. An example of such layered control system is presented in FIG. 3.

FIG. 3 illustrates one embodiment of the organization of the control system of a prosthetic or orthotic device 100. This organization of software processes, data streams, and a priori knowledge is referred to herein as a multi-layer hierarchical architecture. This method of architectural organization can satisfy at least two objectives, namely provide high performance control of an active prosthetic or orthotic device, and efficiently organize processes and data stream(s) such that information received from the sensors of the prosthetic or orthotic device 100 propagates through the system in an orderly and logical fashion.

In some embodiments, the control system of the prosthetic or orthotic device 100 may be or may include a multi-layered controller 300 with three or more layers. For example, the learning layer 110, the inference layer 120, and the reactive layer 130 in FIG. 3 depict the hierarchical layering of the control system architecture of a prosthetic or orthotic device 100. Moreover, the prosthetic or orthotic device 100 and environment 150 highlight the interactions between the control system and the environment in which the prosthetic or orthotic device 100 operates. In some embodiments, the prosthetic or orthotic device 100 includes external interfaces 140 that may be connected to the control system. In some embodiments, the external interfaces 140 may include one or more sensors 142 and/or one or more actuators 144.

In certain embodiments, the control system is organized into hierarchical layers based on an examination of the flow of data into and out of the layers, as well as the interdependencies between layers. The layer names, data abstraction models, and nature of the data stream associated with certain embodiments are described herein using physiological terms. The three layers that can be used to sustain active operation of the prosthetic or orthotic device 100 in certain embodiments include, but are not limited to, a learning layer 110, an inference layer 120, and a reactive layer 130. The different layers can be categorized based on the level of abstraction, the time frame in which they act, etc.

The various layers interact with each other to improve the performance of the prosthetic or orthotic device 100. For example, a well-tuned prosthetic or orthotic device may provide reasonable performance using (only) the reactive layer 130 and the inference layer 120 but may be unable to evolve to meet the user's changing expectations or respond to long term changes in the operating environment without the use of a learning layer 110. In some embodiments, the users of the prosthetic or orthotic device 100 may include an end-user (e.g., an amputee), a clinician or clinical staff member, and/or a manufacturer or manufacturing staff member.

The learning layer 110 can include the control structure's highest abstraction level and can be the level furthest away from raw sensor data. The learning layer 110 can be loosely analogized to human cognitive functions, and it can be used to recursively improve the performance of the control system of the prosthetic or orthotic device 100 as time passes. In addition, the learning layer 110 can have the longest time frame in which it acts. For example, in certain embodiments, the learning layer 110 does not respond to changes in gait pattern between steps. Rather, the learning layer 110 identifies and responds to long-term trends in user performance.

The learning layer 110 can also, or alternatively, define what is considered an improvement in performance. The criteria for improved or reduced performance can evolve with time as the user becomes more familiar with the prosthetic or orthotic device 100 and demands a higher level of performance. In some embodiments, this evolution may be thought of as the transition from a prosthetic or orthotic device possessing a moderate level of performance coupled with a very high level of safety to a prosthetic or orthotic device possessing a high level of performance coupled with reduced user safety constraints, thereby increasing the flexibility and performance potential of the prosthetic or orthotic device. As it evolves, the learning layer 110 can also decide to allow user access to features that had previously been hidden or were not made available. In some embodiments, the user may select different criteria for the learning layer 110 to prioritize. For example, the user may instruct the learning layer 110 to prioritize safety, performance, and/or battery life.

In certain embodiments, the learning layer 110 can include an expert system consisting of rule and data sets that can make decisions as to how well the prosthetic or orthotic device 100 performs in particular situations (e.g., walking, climbing steps, standing up, etc.) and environments (e.g., inside, outside, rainy, etc.) and use the inference layer 120 to implement the high-level decision making. The learning layer 110 alters the rule set or parameter values used by the inference layer 120 incrementally and over longer periods of time to dictate the performance of the prosthetic or orthotic device 100.

In addition, the learning layer 110 can provide support as the controller formulation itself may sometimes change. For example, a prosthetic or orthotic device 100 can potentially cover a large and diversified range of locomotion activities and the user can progressively adapt its behavior, which may rely on additional adjustment to enable the system to maintain a stable level of performance.

The inference layer 120 can be responsible for a variety of different activities. These activities can include, but are not limited to, identifying the current activity or gait phase being performed by the user, predicting future activity or gait phase performed by the user, measuring the performance of the prosthetic or orthotic device 100, requesting that the learning layer 110 examine a particular performance issue, organizing and passing the requested/required data to the learning layer 110, providing the reactive layer 130 with enough data to smoothly execute the task at hand, determining the basic phase of the prosthetic or orthotic device 100 (for example, standing, walking, etc.), determining whether to select active mode or passive mode, etc.

Within the control system, the role of the inference layer 120 can be conceived of as mimicking the human brain's conscious decision-making process. Using the rules and data it acquires from the learning layer 110, the inference layer 120 can infer which course of action is appropriate from sensory data measurements and estimates. Thus, the inference layer 120 can mimic the human ability to apply what one has learned to specific present and future situations.

Unlike the learning layer 110, the inference layer 120 reacts over the course of a single gait cycle. The inference layer 120 can quickly apply rules it has learned from the learning layer 110 to situations as they arise and respond accordingly. For example, the response time of the inference layer 120 can be on the order of tenths of seconds. In some embodiments, the inference layer 120 apply rules over multiple gait cycles. For example, the inference layer 120 can modify the operation of the prosthetic or orthotic device 100 if it detects a change in environment or condition of the user (e.g., fatigue, injury, etc.).

Contextually, the inference layer 120 can be thought of as an intermediate data abstraction level, where the work includes extracting features from an input stream comprising pre-processed data. The data can be processed and characterized to achieve objectives including, but not limited to, managing the prosthetic or orthotic device 100 such that system behavior matches the activity being undertaken by the user and quantifying system performance in terms of overall functionality of the prosthetic or orthotic device 100.

The intermediate position the inference layer 120 occupies within the hierarchy can serve an additional purpose: protecting the reactive layer 130 from the learning layer 110. That is, the inference layer 120 can prevent the learning layer 110 from changing parameters directly used by the reactive layer 130, which may lead to system instability. Instead, the learning layer 110 can direct the inference layer 120 to take a particular action or slightly alter a variable in a data set.

The reactive layer 130, in certain embodiments, represents the lowest abstraction level of the multi-layered controller 300. The reactive layer 130 can directly enforce the desired behavior that is responsive to the activities undertaken by the user and the current environment of the user. Here, the desired behavior can be determined by a rule set of the inference layer 120, which the learning layer 110 can alter. In other words, similar to the general behavior of the human arc-reflex, the reactive layer 130 of certain embodiments can immediately enforce a predefined behavior based on a reduced set of sensory input and estimates. The time frame for the operation of the reactive layer 130 can be on the order of milliseconds or less. More specifically, the reactive layer 130 can handle the motor control laws of the prosthetic or orthotic device 100.

Although the control system architecture presented in FIG. 3 may appear to be hierarchical in nature, the control system architecture can be parallel in structure. For example, the control system may direct the reactive layer 130 to manage itself. For example, in certain embodiments, the reactive layer 130 can use a knee torque sensor output to implement a low-level impedance controller to manage the actuator behavior in real-time. Alternatively, or in addition, the same knee torque sensor output can be used by the inference layer 120 to detect the occurrence of a transition in the user's gait activity to downwards walking. Both decisions present different levels of complexity that can be made independently by the different control layers of the multi-layered controller 300.

Additional details regarding control layers useable with certain embodiments of the invention are disclosed in U.S. Patent Publication No. 2011/0125290 (the “'290 publication”), published May 26, 2011, entitled “Reactive Layer Control System for Prosthetic and Orthotic Devices,” which describes various embodiments and features related to prosthetic or orthotic devices and which is hereby incorporated herein by reference in its entirety.

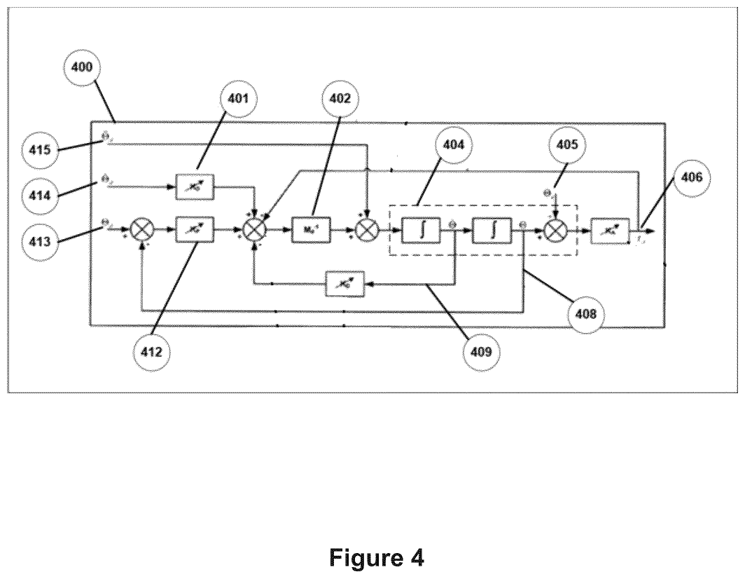

FIG. 4 illustrates a block diagram of one embodiment of an impedance controller 400 of the prosthetic or orthotic device 100. The impedance controller 400 can be implemented at the reactive layer 130. The impedance controller 400 allows the prosthetic or orthotic device 100 to manage the actuator 228 or support the implementation of various behaviors by the actuator 228. For example, the impedance controller 400 may determine the torque to be generated by the actuator to create a predetermined dynamic response or behavior. In some embodiments, the predetermined dynamic actuator response can be determined based on the gait activity, phase and subphase information at the inference layer 120 level.

The impedance controller 400 may be formulated around the combined actuator and knee prosthesis dynamic model 404, to which an external input 405 is used to represent the user interaction with the physical device, represented as a torque or position perturbation added to actuator and prosthetic system dynamic response. In a general and non-limitative manner, the prosthetic or orthotic device 100 is represented as a double integrator. Full implementation of the impedance controller 400 makes use of position feedback 408, velocity feedback 409, and/or force feedback 406 which may be provide as feedback signals. Feedback signals can be measured using physical sensors (e.g., encoders, load cells, torque sensors, IMUs, etc.), or estimated based on observation of other physical quantities (e.g., motor current for torque, output position for velocity, etc.).

The position feedback 408, the velocity feedback 409, and/or force the feedback 406 may be compared to the setpoint signals for position 413, velocity 414 and/or force/acceleration 415 as part of the feedback controller formulation. While the gains can define the dynamic response of the controlled system, the setpoint signals may define a kinematic or kinetic trajectory for the system to follow, additionally to the dynamic response. Time-varying or constant set-points can be used, creating either a tracking controller or a regulator, accordingly to the specific control problem at hand. Additionally, time-varying or constant gains can be used.

In the context of use related to a powered prosthetic device actuator control, a regulator formulation of the control loop is advantageously used for energy dissipative tasks. For example, regulating the knee velocity while yielding under the user weight in stance phase of stairs descent. A tracking controller implementation where the set-point is dynamically varied is advantageously used in power generative tasks, where the knee joint is to be changed angle under the user's weight. For example, when the user is performing the sit-to-stand transition and the knee joint is to extend under the user's weight.

Due to the variable nature of the knee's role across the gait activities, phases and subphases, it is advantageous for the control system of the prosthetic or orthotic device 100 to change the knee joint actuator behavior accordingly. The capacity to dynamically adapt the impedance controller gains based on the phase, subphases and gait activity can be advantageously leveraged to create a simple mechanism to adapt the actuator behavior while maintaining a single controller implementation.

It should be understood that while it can be beneficial to use all the gains and/or set-points signals introduced above simultaneously, in many cases the gait tasks requirements will not require the use of all terms or features of the impedance controller 400. Since the impedance controller 400 combines three simpler control loops, independent use of any of these control loop or any combination of these is possible and desirable in some circumstances.

In some cases, the prosthetic or orthotic device 100 may use the impedance controller 400 to define the actuator dynamic behavior as passive. For example, based on the inference layer 120 determining that the user of the prosthetic or orthotic device 100 is standing, a command can be issued to the reactive layer 130 to enter a passive mode. During the passive mode, the actuator can exhibit a force rejection behavior during some of or the entire stance phase and a force following behavior during some of or the entire swing phase. The force rejection behavior and/or the force following behavior may be implemented by defining the impedance controller position 412, velocity 401 and mass 402 gains at the appropriate values. These gains can be empirically tuned through series of experiments or calculated based on the desired dynamic response and the prosthetic system characteristics.

In some cases, the prosthetic or orthotic device 100 may use the impedance controller 400 to define the actuator behavior as active. For example, based on the inference layer 120 determining that the user of the prosthetic or orthotic device 100 is walking, the inference layer 120 can communicate a command to a reactive layer 130 to enter an active mode. During the active mode, the impedance controller 400 can cause the actuator to exhibit a force rejection behavior and a toe-off assist behavior during the stance phase. For example, during the active mode, the actuator can exhibit the force rejection behavior between heel-strike and midstance and the toe-off assist behavior between midstance and toe-off. The force rejection behavior and the toe-off assist behavior can be defined through definition of the appropriate gain and set-point values. As described above, the gain and set-point values can be empirically tuned through series of experiments or calculated based on the desired dynamic response and the prosthetic system characteristics. In some embodiments, the learning layer 110 can adjust the gain and set-point values to improve the performance of the force rejection behavior, the force following behavior, and the toe-off assist behavior.

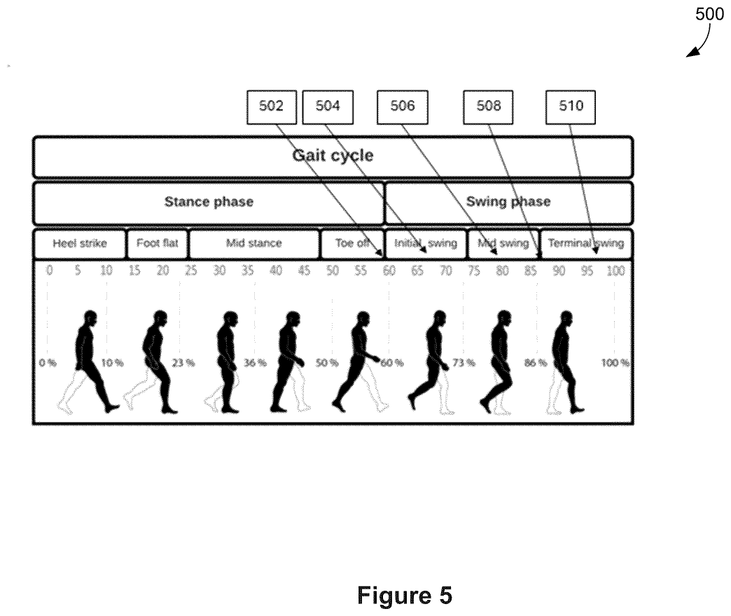

FIG. 5 illustrates a diagram of a gait cycle 500 of a prosthetic or orthotic device (e.g., the prosthetic or orthotic device 100 as described herein). As discussed herein, the gait cycle 500 (e.g., a gait cycle for a walking locomotion activity) may include one or more phases (e.g., a swing phase and a stance phase). For example, during a stance phase for a prosthetic or orthotic device, at least a portion of a foot associated with the prosthetic or orthotic device may be in contact with a ground surface and, during a swing phase for the prosthetic or orthotic device, the prosthetic or orthotic device may perform a swinging motion and the at least a portion of the foot may not be in contact with the ground surface for all or a portion of the swing phase.

All or a portion of the one or more phases may include one or more sub-phases. For example, a stance phase may include a heel strike portion (e.g., during which a heel of a foot strikes the ground), a foot flat portion (e.g., during which the foot is flat against a ground surface), a mid-stance portion (e.g., during which the prosthetic or orthotic device is oriented in a stance position), and a toe-off portion (e.g., during which the foot is used to push away from the ground). The swing phase may include an initial swing portion (e.g., during which the prosthetic or orthotic device may perform a flexion action), a mid-swing portion (e.g., during which the prosthetic or orthotic device switches from performance of the flexion action to performance of an extension action), and a terminal swing portion (e.g., during which the prosthetic or orthotic device performs the extension action and completes the swing phase).

All or a portion of the one or more phases and/or the one or more sub-phases may correspond to a subset (e.g., a temporal subset) of the gait cycle. For example, the stance phase may correspond to 60% of the gait cycle and the swing phase may correspond to 40% of the gait cycle. In another example, the heel strike portion may correspond to 15% of the gait cycle, the foot flat portion may correspond to 10% of the gait cycle, the mid-stance portion may correspond to 23% of the gait cycle, the toe-off portion may correspond to 12% of the gait cycle, the initial swing portion may correspond to 15% of the gait cycle, the mid-swing portion may correspond to 10% of the gait cycle, and the terminal swing portion may correspond to 15% of the gait cycle.

All or a portion of the one or more sub-phases may be associated with one or more actions and/or one or more parameters (e.g., one or more time varying parameters, one or more dynamic parameters, etc.). For example, all or a portion of the one or more sub-phases may be associated with one or more actions for performance by the prosthetic or orthotic device. In another example, all or a portion of the one or more sub-phases may be associated with one or more parameters (e.g., one or more gains) of a controller for performance of the one or more actions by the prosthetic or orthotic device.

The toe-off portion may be associated with a toe-off assist action 502 (e.g., a toe-off assist function). For example, performance of the toe-off assist action 502 by the prosthetic or orthotic device may cause the prosthetic or orthotic device to transition from the toe-off portion to the initial swing portion and may propel a user forward (e.g., during a walking locomotion activity). In another example, the toe-off assist action 502 may provide flexion assistance to propel a user forward. To perform the toe-off assist action 502, a control system of the prosthetic or orthotic device may cause the prosthetic or orthotic device to provide a force to a ground surface and push off of the ground surface such that the user is propelled forward. In some cases, to perform the toe-off assist action 502, the control system may provide a control signal to an actuator of the prosthetic or orthotic device that may cause the actuator to actuate. Actuation of the actuator may cause the prosthetic or orthotic device to initiate a flexion action (e.g., such that a joint of the prosthetic or orthotic device moves towards a 90 degree angle).

All or a portion of the initial swing portion, the mid-swing portion, and the terminal swing portion may be associated with one or more swing actions 504, 506, and 508. In the example of FIG. 5, the initial swing portion is associated with a swing action 504, the mid-swing portion is associated with a swing action 506, and the mid-swing portion and the terminal swing portion are associated with a swing action 508. For example, performance of the one or more swing actions 504, 506, and 508 by the prosthetic or orthotic device may cause the prosthetic or orthotic device to continue performance of the flexion action and/or an extension action. To perform the one or more swing actions 504, 506, and 508, the control system may provide a control signal to the actuator that may cause the actuator to dynamically adjust the torque (e.g., the torque gain) provided by the actuator. For example, the control system may cause the actuator to provide a torque (e.g., a high torque gain) during the initial swing portion and may decrease (e.g., progressively reduce over time) the provided torque during the initial swing portion (e.g., during performance of the initial swing portion of the swing phase) to slow the prosthetic or orthotic device down.

In some cases, performance of the one or more swing actions 504, 506, and 508 may cause the prosthetic or orthotic device to perform a flexion action to move towards a target flexion angle (e.g., a maximum flexion angle) of a joint of the prosthetic or orthotic device and to perform an extension action to move towards a target extension angle of the joint. For example, performance of the one or more swing actions 504, 506, and 508 may cause the prosthetic or orthotic device to perform the flexion action to move the angle of the joint to the target flexion angle and the prosthetic or orthotic device may determine that the angle corresponds to the target flexion angle prior to initiating performance of the extension action.

In some cases, the swing phase may not be associated with separate force following behaviors. For example, instead of implementing a first force following behavior for performance of the flexion action and a second force following behavior for performance of an extension action, the prosthetic or orthotic device may implement one action and/or behavior for performance of the flexion action and the extension action.

The terminal swing portion may be associated with a brake action 510. For example, performance of the brake action 510 by the prosthetic or orthotic device may cause the prosthetic or orthotic device to brake the actuator and reduce the torque provided by the actuator. To perform the brake action 510, the control system may provide a control signal to the actuator that may cause the actuator to reduce the amount of torque provided by the actuator. In some cases, the control system may cause the actuator to maintain a fixed position (e.g., corresponding to a particular angle). For example, the control system may cause the actuator to maintain a fixed position associated with a stance phase for the prosthetic or orthotic device based on an angle of the joint such that the foot contacts a ground surface with the actuator at the fixed position.

In some cases, the brake action 510 may correspond to a bumper avoidance behavior. For example, the bumper avoidance behavior may correspond to a braking behavior associated with full extension of the prosthetic or orthotic device (e.g., a behavior to brake the prosthetic or orthotic device as the prosthetic or orthotic device approaches full extension).

In some cases, to perform one or more of the toe-off assist action 502, the one or more swing actions 504, 506, and 508, and/or the brake action 510, the prosthetic or orthotic device may obtain sensor data (e.g., indicative of an angle, a velocity, etc. associated with the prosthetic or orthotic device). For example, the prosthetic or orthotic device may include one or more sensors (e.g., an angle sensor, a proximity sensor, a position sensor, accelerometers, etc.) and may obtain the sensor data from the one or more sensors. The prosthetic or orthotic device may perform one or more of the toe-off assist action 502, the one or more swing actions 504, 506, and 508, and/or the brake action 510 based on comparing the sensor data to a threshold.

In some cases, the sensor data may include sensor data associated with a stance phase and/or a swing phase of the same gait cycle and the prosthetic or orthotic device may use the sensor data associated with the stance phase and/or the swing phase to control the prosthetic or orthotic device during the swing phase of the same gait cycle. For example, the sensor data may be indicative of a velocity associated with the prosthetic or orthotic device (e.g., a velocity of a user, a velocity of a thigh of the user, a velocity of a thigh portion of the prosthetic or orthotic device, etc.).

In some cases, the prosthetic or orthotic device may use the sensor data to transition between a flexion action and an extension action. For example, the prosthetic or orthotic device may determine an angle associated with the prosthetic or orthotic device based on the sensor data. The prosthetic or orthotic device may determine that the angle satisfies a threshold (e.g., 135 degrees, 130 degrees, 120 degrees, etc.). Based on determining that the angle satisfies the threshold, the prosthetic or orthotic device may adjust the torque provided by the actuator (e.g., to slow the angular velocity of the prosthetic or orthotic device) prior to transitioning from the flexion action to the extension action. By transitioning from the flexion action to the extension action in this manner, the prosthetic or orthotic device can preemptively prepare for the transition and smoothly perform the transition.

In some cases, the prosthetic or orthotic device may use the sensor data to determine a torque to cause the actuator to provide during performance of the flexion action. For example, the prosthetic or orthotic device may use the sensor data to determine a parameter for the controller which, in turn, may affect the torque provided by the actuator. The prosthetic or orthotic device may determine a velocity (e.g., a rotational velocity) associated with the prosthetic or orthotic device based on the sensor data (e.g., a velocity of a thigh of the user during a swing phase of the gait cycle) and may determine a parameter of the controller based on the determined velocity. For example, the prosthetic or orthotic device may correlate the parameter of the controller and the velocity such that the prosthetic or orthotic device may increase the parameter of the controller based on a determined velocity associated with a user. By correlating the velocity to the parameter of the controller, the prosthetic or orthotic device can adapt, in real time, to a velocity associated with a user.

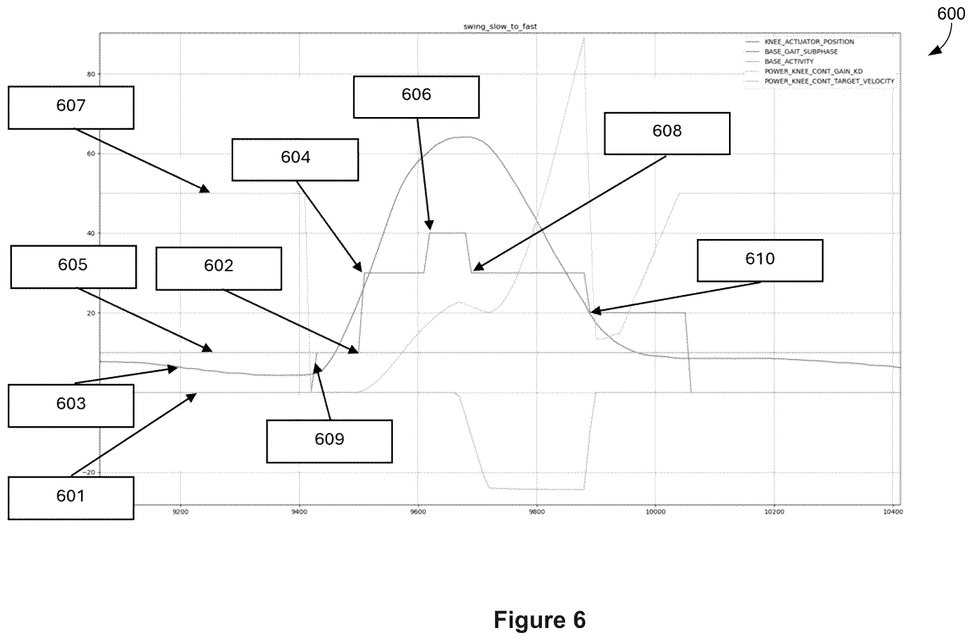

FIG. 6 illustrates a diagram 600 of various swing dynamics for a gait cycle of a prosthetic or orthotic device (e.g., the prosthetic or orthotic device 100 as described herein). The prosthetic or orthotic device may include one or more actuators that may actuate to cause movement by the prosthetic or orthotic device and a control system (e.g., one or more controllers) that may provide control signals to the actuator. Further, the one or more controllers may include data processing hardware and/or memory hardware.

The control system may provide a control signal based on the below Equation 1.

$$u ( t ) = {K}_{p} e ( t ) + {K}_{d} \frac{d}{dt} e ( t ) + {K}_{m} τ \qquad Equation 1$$

In Equation 1, Kₚ, K_{d}, and Kₘ may be the gains (e.g., the controller gains), e(t) may be an error (e.g., an error of a sensor value), τ may be a moment (e.g., a moment of the actuator), and u(t) may be the control signal. In some cases, the prosthetic or orthotic device may determine (e.g., may measure) e(t) and/or t (e.g., based on sensor data). To provide a smoother operation of the prosthetic or orthotic device, the control system may dynamically adjust one or more of the gains (e.g., during, prior to, or subsequent performance of a gait cycle).

The swing dynamics include a target velocity 601, an actuator position 603, a base activity 605, a gain (e.g., K_{d}) of a controller 607 of the prosthetic or orthotic device, and a gait cycle 609. The diagram plots a value of the swing dynamics (e.g., with respect to the y-axis) over time (e.g., with respect to the x-axis). The diagram may plot a value of the swing dynamics for all or a portion of the swing dynamics for all or a portion of a gait cycle (e.g., for a swing phase of a gait cycle).

As illustrated in FIG. 6, the gait cycle 609 includes a plurality of sub-phases. For example, the gait cycle 609 includes a toe-off portion 602, an initial swing portion 604, a mid-swing portion 606 and 608, and a terminal swing portion 610. A change in a value of the gait cycle 609 may indicate a transition between portions of the gait cycle 609 (e.g., a transition from the toe-off portion to the initial swing portion).

As discussed herein, during the toe-off portion 602, the prosthetic or orthotic device may perform a toe-off assist action. To perform the toe-off assist action, the prosthetic or orthotic device may provide a force to a ground surface via a foot associated with the prosthetic or orthotic device. The force may cause the foot of the prosthetic or orthotic device to lift from the ground surface.

During the initial swing portion 604 and all or a portion of the mid-swing portion 606 and 608, the prosthetic or orthotic device may reduce the torque provided by the actuator. In some cases, the prosthetic or orthotic device may reduce the torque (e.g., continuously) from performance of a toe-off assist action to completion of the flexion action (e.g., achieving maximum flexion). Reducing the torque provided by the actuator may cause a reduction in a velocity (e.g., a knee angular velocity) associated with the prosthetic or orthotic device. For example, reducing the torque provided by the actuator may cause the prosthetic or orthotic device to slow down due to gravity. In some cases, the prosthetic or orthotic device may reduce the torque provided by the actuator such that the velocity associated with the prosthetic or orthotic device approaches and/or equals 0 degrees per second.

The prosthetic or orthotic device may decrease and/or increase the gain (e.g., during performance of the gait cycle). For example, as shown in FIG. 6, during the initial swing portion 604 and a first subset of the mid-swing portion 606 and 608, the prosthetic or orthotic device may increase the gain of the controller 607. During a second subset of the mid-swing portion 606 and 608, the prosthetic or orthotic device may decrease the gain of the controller 607. In some cases, the prosthetic or orthotic device may decrease and/or increase the gain during performance of the flexion action and/or the extension action. For example, the prosthetic or orthotic device may perform a plurality of decreases and/or increases to the gain during performance of the flexion action and/or the extension action. The prosthetic or orthotic device may increase the gain and subsequently decrease the gain (e.g., a smaller decrease as compared to the increase) during performance of the flexion action. By increasing and/or decreasing the gain of the controller, the prosthetic or orthotic device may provide for a smoother experience during performance of the gait cycle.

In some cases, the prosthetic or orthotic device may decrease and/or increase the gain according to different rates of adjustment and/or for different time periods. For example, the prosthetic or orthotic device may increase the gain according to a first rate of adjustment (e.g., a 10% increase per second) and subsequently decrease the gain according to a second rate of adjustment (e.g., a 2% decrease per second) during performance of the flexion action.

The prosthetic or orthotic device may obtain sensor data associated with the prosthetic or orthotic device. Based on the sensor data, the prosthetic or orthotic device may determine a position, an orientation, an angle, a speed, etc. associated with the prosthetic or orthotic device. For example, the prosthetic or orthotic device may determine an angle formed by a first segment of the prosthetic or orthotic device and a second segment of the prosthetic or orthotic device (e.g., the first and second segments connected via a joint of the prosthetic or orthotic device). In another example, the prosthetic or orthotic device may determine an angle formed by a segment of the prosthetic or orthotic device and a user (e.g., a thigh of a user).

The prosthetic or orthotic device may obtain (e.g., identify) a threshold (e.g., a threshold range, a threshold value, etc.) and may compare the sensor data (e.g., a value indicated by the sensor data) to the threshold. For example, the threshold may indicate a threshold angle and the prosthetic or orthotic device may compare an angle as indicated by the sensor data to the threshold angle.

Based on comparing the sensor data to the threshold, the prosthetic or orthotic device may determine whether the sensor data satisfies (e.g., is greater than, is less than, matches, is within a particular range of, etc.) the threshold. For example, the prosthetic or orthotic device may determine that the sensor data indicates the prosthetic or orthotic device is associated with a 160 degree angle and may determine that the angle does not satisfy a threshold angle (e.g., a 135 degree angle).

In some cases, the prosthetic or orthotic device may increase the gain of the controller based on determining that the sensor data satisfies the threshold. For example, during performance of the flexion action by the prosthetic or orthotic device, the prosthetic or orthotic device may increase the gain of the controller based on determining that the angle of the prosthetic or orthotic device satisfies a threshold angle to brake the prosthetic or orthotic device until the prosthetic or orthotic device is stopped. The prosthetic or orthotic device may increase the gain of the controller to brake movement of the prosthetic or orthotic device as an angle associated with the prosthetic or orthotic device approaches a maximum flexion angle (e.g., a 90 degree angle). For example, the prosthetic or orthotic device may determine a time period for the angle of the prosthetic or orthotic device reaching a maximum flexion angle based on an angle, velocity, torque, etc. associated with the prosthetic or orthotic device and may increase the gain based on the determined time period.

The prosthetic or orthotic device may determine performance of the flexion action and initiation of performance of the extension action (e.g., that the prosthetic or orthotic device is moving into extension). In some cases, based on determining that the sensor data satisfies a second threshold, the prosthetic or orthotic device may determine that the prosthetic or orthotic device has completed the flexion action (e.g., has achieved maximum flexion) and is initiating performance of the extension action.

Based on determining initiation of the performance of the extension action, the prosthetic or orthotic device may determine a gain (e.g., a value for the gain) for the controller for performance of the extension action. In some cases, the prosthetic or orthotic device may determine a velocity associated with the prosthetic or orthotic device based on the sensor data and may determine (e.g., perform a real time adjustment of) the gain and/or the torque (e.g., a motorized extension assistance level) based on the velocity. For example, the prosthetic or orthotic device may correlate the velocity and the gain such that an increase in the velocity correlates to an increase in the gain. By correlating the velocity and the gain, the prosthetic or orthotic device may provide a smoother extension action. In some cases, the prosthetic or orthotic device may gradually increase the gain as the prosthetic or orthotic device performs the extension action. For example, the prosthetic or orthotic device may gradually increase the motorized extension assistance level as the prosthetic or orthotic device performs the extension action.

In some cases, the prosthetic or orthotic device may determine the velocity of the prosthetic or orthotic device during the swing phase (e.g., as compared to determining the velocity of the prosthetic or orthotic device during a different (earlier) swing phase). For example, the prosthetic or orthotic device may determine the velocity of the prosthetic or orthotic device in real time.

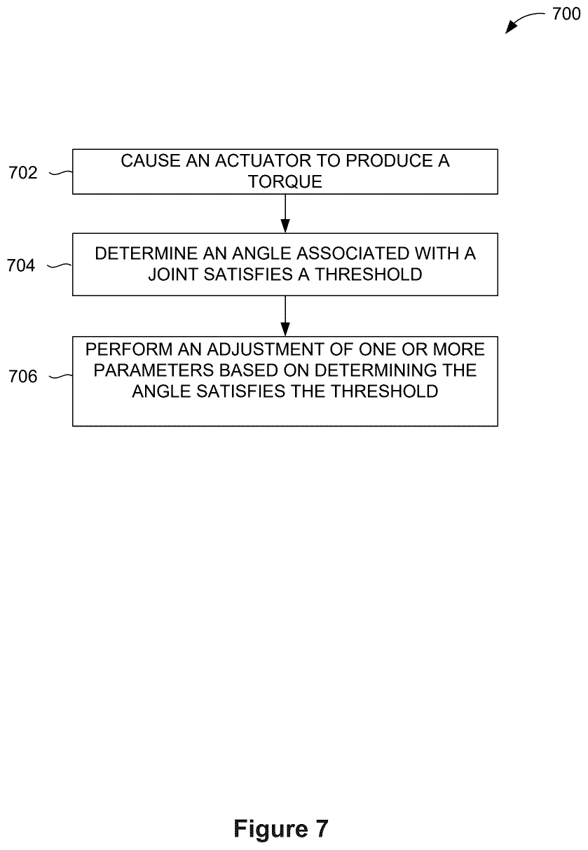

FIG. 7 is a flow diagram 700 depicting an example arrangement of operations for operation of a prosthetic or orthotic device (e.g., a motorized prosthetic or orthotic device). The prosthetic or orthotic device may include a first segment (e.g., a thigh segment), a second segment (e.g., a lower leg segment), a joint (e.g., a knee joint) coupling the first segment and the second segment, an actuator coupled to the first segment and the second segment and controlling motion of the second segment relative to the first segment, and a control system (e.g., one or more controllers, data processing hardware, memory hardware, one or more sensors, etc.). For example, the memory hardware may store computer-executable instructions and execution of the instructions by the data processing hardware may cause performance of the operations described herein. In some cases, the first segment may be a proximal connector that attaches the prosthetic or orthotic device to a thigh of a trans-femoral amputee. In some cases, the prosthetic or orthotic device may include a motorized knee.

In some cases, the control system may determine initiation of a swing phase of a locomotion activity. For example, the control system may determine initiation of a swing phase of a gait cycle of a walking locomotion activity.

The swing phase may include performance of a flexion action and an extension action. The flexion action may be associated with a first time period and the extension action may be associated with a second time period. In some cases, the swing phase may be disproportional (e.g., non-symmetrical). For example, the second time period may exceed the first time period such that the progression of an angle of the prosthetic or orthotic device is asymmetrical.

In some cases, the locomotion activity and/or the prosthetic or orthotic device may be associated with a stance phase and/or a swing phase. The stance phase may include a heel strike portion, a foot flat portion, a mid-stance portion, and a toe-off assist portion. The swing phase may include an initial swing portion, a mid-swing portion, and a terminal swing portion.

At 702, the control system causes an actuator to produce a torque (e.g., a first torque). For example, the control system may cause the actuator to produce a torque based on one or more parameters (e.g., one or more gains) of a controller. In some cases, the control system may cause the actuator to produce the torque based on determining initiation of the swing phase. The prosthetic or orthotic device (e.g., a motorized knee of the prosthetic or orthotic device) may perform at least a first portion of a flexion action based on the first torque. For example, the motorized knee may initiate performance of the flexion action. In some cases, the control system may progressively reduce the first torque during performance of the flexion action.

In some cases, the prosthetic or orthotic device may perform the flexion action to move an angle of a joint of the prosthetic or orthotic device to a target flexion angle (e.g., a maximum flexion angle) and may perform an extension action to move an angle of the joint from the target flexion angle to a target extension angle. For example, the target flexion angle may be 90 degrees. In some cases, the target flexion angle may be walking speed generic. For example, the target flexion may be the same for different walking speeds of a user.

In some cases, the prosthetic or orthotic device may obtain an input defining and/or indicating the target flexion angle. For example, the prosthetic or orthotic device may obtain an input from a user computing device and may determine the target flexion angle based on the input.

In some cases, the control system may cause the actuator to produce the torque based on one or more parameters of the control system. In some cases, the control system may perform an adjustment of the one or more parameters of the control system and may cause the actuator to produce the torque based on the adjustment of the one or more parameters. For example, the adjustment may be a gradual adjustment of the one or more parameters over time.

At 704, the control system determines an angle (e.g., a first angle) associated with a joint satisfies a threshold (e.g., a first threshold). For example, the control system may obtain sensor data from one or more sensors and may determine the angle based on the sensor data. In some cases, the angle may be and/or may include an angle formed by the first segment and the second segment. In some cases, the angle may be and/or may include formed by the user (e.g., a thigh of the user) and the second segment.

At 706, the control system performs an adjustment (e.g., a first adjustment) of one or more parameters (e.g., of the controller) based on determining the angle satisfies the threshold. For example, the adjustment may be a gradual increase to one or more values of the one or more parameters. In some cases, the control system may perform the adjustment based on (e.g., in response to) performance of a toe-off assist action by the prosthetic or orthotic device. The control system may cause the actuator to produce a second torque based on the adjustment. The prosthetic or orthotic device may perform at least a second portion of a flexion action based on the second torque.

In some cases, the control system may control the actuator based on the adjustment. For example, the control system may provide a control signal to the actuator based on the adjustment.

In some cases, the adjustment may be a progressive adjustment of the one or more parameters over time. For example, the prosthetic or orthotic device may perform at least a portion of the flexion action and/or at least a portion of the extension action during the progressive adjustment of the one or more parameters. In some cases, the control system may continuously adjust the one or more parameters during performance of at least a portion of the flexion action and/or at least a portion of the extension action. In some cases, the flexion action and/or the extension action may be associated with different rates of adjustment of the one or more parameters (e.g., a first rate of adjustment during a first time period, a second rate of adjustment during a second time period, etc.).

In some cases, the control system may determine a velocity associated with the prosthetic or orthotic device (e.g., a velocity of a user, a rotational velocity of the first segment, etc.). For example, the control system may obtain sensor data and may determine the velocity based on the sensor data. In some cases, the velocity may be a velocity associated with a thigh of a user.

The control system may determine that the velocity satisfies a threshold (e.g., a second threshold). Based on determining the velocity and/or determining that the velocity satisfies the threshold, the control system may perform a second adjustment of the one or more parameters. The control system may cause the actuator to produce a third torque based on the second adjustment. The prosthetic or orthotic device may perform at least a portion of an extension action based on the third torque.

In some cases, the control system may determine performance of a first portion of the extension action by the prosthetic or orthotic device. The control system may perform the second adjustment based on determining the performance of the first portion of the extension action by the prosthetic or orthotic device. In response to the second adjustment, the prosthetic or orthotic device may perform at least a second portion of the extension action.

In some cases, the control system may perform the same adjustment to the one or more parameters for different locomotion activities, different gait cycles, different walking speeds, etc. For example, the control system may determine initiation of a swing phase of a first locomotion activity associated with a first velocity. The swing phase of the first locomotion activity may include performance of the flexion action and performance of the extension action. The control system may perform the first adjustment based on determining the initiation of the swing phase of the first locomotion activity. The control system may determine initiation of a swing phase of a second locomotion activity associated with a second velocity and may perform (e.g., reperform, perform again, etc.) the first adjustment based on determining the initiation of the swing phase of the second locomotion activity.

In some cases, the control system may utilize the same target flexion angle for different locomotion activities, different gait cycles, different walking speeds, etc. For example, a user may be walking with a first walking speed and the control system may cause the prosthetic or orthotic device to perform the flexion action to flex to the target flexion angle and (e.g., subsequently) a user (or a different user) may be walking with second, different walking speed and the control system may cause the prosthetic or orthotic device to perform the flexion action to flex to the same target flexion angle.

In some cases, the control system may determine a second angle associated with the joint satisfies a second threshold. The control system may perform a second adjustment of the one or more parameters based on determining the second angle satisfies the second threshold. The control system may cause the prosthetic or orthotic device to perform the terminal swing portion based on the second adjustment. In some cases, the second adjustment may cause the actuator to maintain a fixed position.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise. Additionally, as used herein, “gradually” has its ordinary meaning (e.g., differs from a non-continuous, such as a step-like, change).

The scope of the present disclosure is not intended to be limited by the specific disclosures of preferred embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A prosthetic or orthotic device comprising:
a first segment;
a second segment coupled to the first segment at a joint;
an actuator coupled to the first segment and the second segment and configured to control motion of the second segment relative to the first segment; and
a controller configured to:
cause an actuator to produce a first torque based on one or more parameters of the controller, wherein a first portion of a flexion action is performed by the prosthetic or orthotic device based on the first torque, wherein the first torque progressively reduces during performance of the flexion action;
determine a first angle associated with the joint satisfies a first threshold; and
perform a first adjustment of the one or more parameters based on determining the first angle satisfies the first threshold and performance of a toe-off assist action, wherein the controller causes the actuator to produce a second torque based on the first adjustment, and wherein a second portion of the flexion action is performed by the prosthetic or orthotic device based on the second torque.

2. The prosthetic or orthotic device of claim 1, wherein the controller is further configured to:
determine a velocity associated with the prosthetic or orthotic device satisfies a second threshold; and
perform a second adjustment of the one or more parameters based on determining the velocity satisfies the second threshold, wherein the controller causes the actuator to produce a third torque based on the second adjustment, and wherein at least a portion of an extension action is performed by the prosthetic or orthotic device based on the third torque.

3. The prosthetic or orthotic device of claim 2, wherein the velocity comprises a rotational velocity of the first segment.

4. The prosthetic or orthotic device of claim 1, wherein the controller is further configured to:
determine a velocity associated with the prosthetic or orthotic device satisfies a second threshold;
determine a first portion of an extension action is performed by the prosthetic or orthotic device; and
perform a second adjustment of the one or more parameters based on determining the velocity satisfies the second threshold and based on determining the performance of the first portion of the extension action, wherein the controller causes the actuator to produce a third torque based on the second adjustment, and wherein a second portion of the extension action is performed by the prosthetic or orthotic device based on the second adjustment.

5. The prosthetic or orthotic device of claim 1, wherein the controller is further configured to:
determine a velocity associated with the prosthetic or orthotic device; and
perform a second adjustment of the one or more parameters based on the velocity, wherein the controller causes the actuator to produce a third torque based on the second adjustment, and wherein at least a portion of an extension action is performed by the prosthetic or orthotic device based on the second adjustment.

6. The prosthetic or orthotic device of claim 1, wherein the controller is further configured to:
determine initiation of a swing phase of a locomotion activity, wherein the swing phase comprises performance of the flexion action and performance of an extension action, and wherein causing the actuator to produce the first torque is based on determining the initiation of the swing phase.

7. The prosthetic or orthotic device of claim 6, wherein the flexion action is associated with a first time period, wherein the extension action is associated with a second time period, and wherein the second time period exceeds the first time period.

8. The prosthetic or orthotic device of claim 1, wherein the one or more parameters comprise one or more gains.

9. The prosthetic or orthotic device of claim 1, wherein the first adjustment comprises a progressive adjustment of the one or more parameters over time, wherein the prosthetic or orthotic device performs at least a portion of a flexion action during performance of the first adjustment.

10. The prosthetic or orthotic device of claim 9, wherein the flexion action is associated with different rates of adjustment of the one or more parameters.

11. The prosthetic or orthotic device of claim 1, wherein the first adjustment comprises a gradual increase to one or more values of the one or more parameters.

12. The prosthetic or orthotic device of claim 1, wherein the controller is further configured to:
determine initiation of a swing phase of a first locomotion activity associated with a first velocity, wherein the swing phase of the first locomotion activity comprises performance of the flexion action and performance of an extension action, and wherein the first adjustment is based on determining the initiation of the swing phase of the first locomotion activity;
determine initiation of a swing phase of a second locomotion activity associated with a second velocity; and
perform the first adjustment based on determining the initiation of the swing phase of the second locomotion activity.

13. The prosthetic or orthotic device of claim 1, wherein the prosthetic or orthotic device is associated with a stance phase and a swing phase, wherein the stance phase comprises a heel strike portion, a foot flat portion, a mid-stance portion, and a toe-off assist portion, wherein the swing phase comprises an initial swing portion, a mid-swing portion, and a terminal swing portion, wherein the controller is further configured to:
determine a second angle associated with the joint satisfies a second threshold; and
perform a second adjustment of the one or more parameters based on determining the second angle satisfies the second threshold, wherein the controller causes the prosthetic or orthotic device to perform the terminal swing portion based on the second adjustment.

14. The prosthetic or orthotic device of claim 13, wherein the second adjustment causes the actuator to maintain a fixed position.

15. The prosthetic or orthotic device of claim 1, wherein the prosthetic or orthotic device further comprises a control system, wherein the control system comprises one or more sensors and the controller, wherein the controller is further configured to:
obtain sensor data from the one or more sensors; and
determine the first angle based on the sensor data.

16. The prosthetic or orthotic device of claim 1, wherein the controller is further configured to:
control the actuator based on the first adjustment.

17. The prosthetic or orthotic device of claim 1, wherein the first segment comprises a proximal connector, wherein the proximal connector is configured to attach the prosthetic or orthotic device to a thigh of a trans-femoral amputee.

18. The prosthetic or orthotic device of claim 17, wherein the controller is further configured to:
determine a velocity associated with the thigh satisfies a second threshold; and
perform a second adjustment of the one or more parameters based on determining the velocity satisfies the second threshold, wherein the controller causes the actuator to produce a third torque based on the second adjustment, and wherein at least a portion of an extension action is performed by the prosthetic or orthotic device based on the second adjustment.

19. A non-transitory computer readable medium having instructions stored thereon, wherein execution of the instructions by a controller causes the controller to:
cause an actuator to produce a torque based on one or more parameters of a controller, wherein a prosthetic or orthotic device comprises a motorized knee, the actuator, and the controller, and wherein the controller causes the motorized knee to perform a first portion of a flexion action based on the torque, wherein the torque progressively reduces during performance of the flexion action;
determine a first angle associated with a knee joint of the motorized knee satisfies a first threshold; and
perform a first adjustment of the one or more parameters based on determining the first angle satisfies the first threshold and performance of a toe-off assist action, wherein the controller causes the motorized knee to perform a second portion of the flexion action based on the first adjustment.

20. A control system, for controlling a prosthetic or orthotic device comprising a controller, a shank segment, and a motorized knee at a knee joint that is configured to attach to attach to a thigh of a trans-femoral amputee, the controller configured to:
cause an actuator to produce a torque based on one or more parameters of the controller, wherein the controller causes the motorized knee to perform a first portion of a flexion action based on the torque, wherein the torque progressively reduces during performance of the flexion action;
determine a first angle associated with a knee joint of the motorized knee satisfies a first threshold; and
perform a first adjustment of the one or more parameters based on determining the first angle satisfies the first threshold and performance of a toe-off assist action, wherein the controller causes the motorized knee to perform a second portion of the flexion action based on the first adjustment.
